(19) United States
(12) Patent Application Publication
(10) Pub. No.: US 2021/0090714 A1
(43) Pub. Date: Mar. 25, 2021

(54) VIRTUAL REALITY BEHAVIORAL HEALTH PLATFORM

(71) Applicant: THERA Inc., Emeryville, CA (US)

(72) Inventors: Erin BOGDANSKI, Emeryville, CA (US); Amit JAIN, Emeryville, CA (US)

(21) Appl. No.: 16/732,857

(22) Filed: Jan. 2, 2020

Related U.S. Application Data
(60) Provisional application No. 62/905,806, filed on Sep. 25, 2019.

(51) Int. Cl. G16H 20/70; G16H 10/60; G16H 40/67; G16H 10/20

(52) U.S. Cl. CPC G16H 20/70; G16H 10/20; G16H 40/67; G16H 10/60

(57) ABSTRACT

A machine has a processor and a network interface circuit connected to the processor and a network. A memory is connected to the processor. The memory stores instructions executed by the processor to process user supplied data to derive a list of therapists. A selected therapist is received from a patient. A session between the patient and the selected therapist is coordinated. The selected therapist specifies virtual reality parameters for the session. The patient specifies at least one avatar for the session. The session is hosted in accordance with the virtual reality parameters, such that the session transpires in a virtual reality environment without identifying the real life visual attributes of the patient or selected therapist, while utilizing audio communications between the patient and the selected therapist.

### CROSS-REFERENCE TO RELATED APPLICATION

This application claims priority to U.S. Provisional Patent Application Ser. No. 62/905,806, filed Sep. 25, 2019, the contents of which are incorporated herein by reference.

### FIELD OF THE INVENTION

This invention relates generally to computer network communications. More particularly, this invention is a virtual reality behavioral health platform.

### BACKGROUND OF THE INVENTION

Many traumatized individuals are reluctant to seek in-person therapy that may improve their health and wellbeing. Consequently, there is an acute need for alternate therapeutic modalities to assist such individuals.

### SUMMARY OF THE INVENTION

A machine has a processor and a network interface circuit connected to the processor and a network. A memory is connected to the processor. The memory stores instructions executed by the processor to process user supplied data to derive a list of therapists. A selected therapist is received from a patient. A session between the patient and the selected therapist is coordinated. The selected therapist specifies virtual reality parameters for the session. The patient specifies at least one avatar for the session. The session is hosted in accordance with the virtual reality parameters, such that the session transpires in a virtual reality environment without identifying the real life visual attributes of the patient or selected therapist, while utilizing audio communications between the patient and the selected therapist.

### BRIEF DESCRIPTION OF THE FIGURES

The invention is more fully appreciated in connection with the following detailed description taken in conjunction with the accompanying drawings, in which:

FIG. 1 illustrates a system configured in accordance with an embodiment of the invention.

FIG. 2 illustrates processing operations associated with an embodiment of the invention.

FIG. 3 illustrates a graphical user interface utilized in accordance with an embodiment of the invention.

Like reference numerals refer to corresponding parts throughout the several views of the drawings.

### DETAILED DESCRIPTION OF THE INVENTION

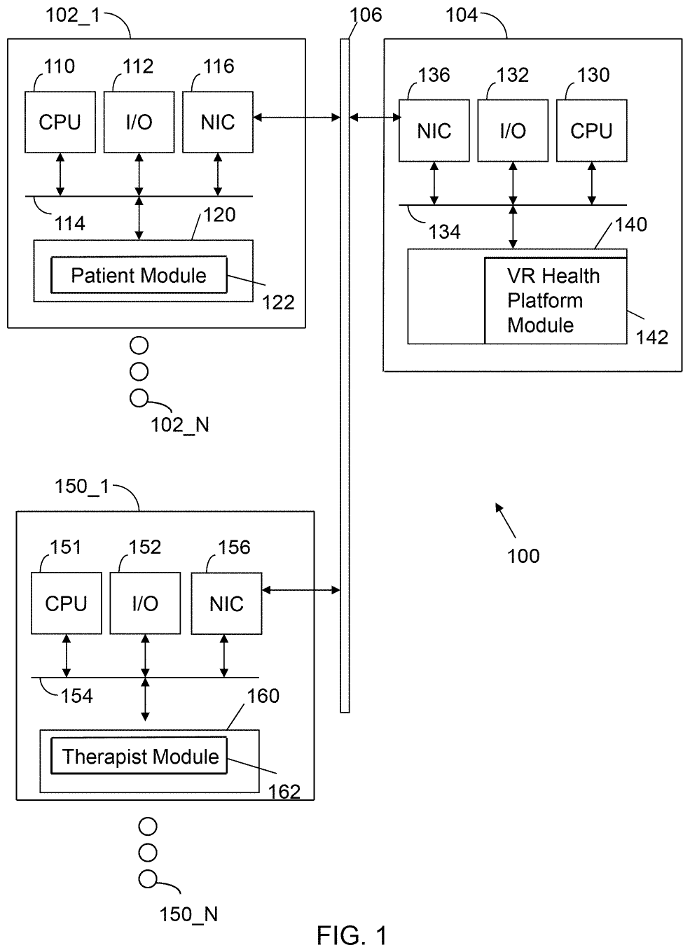

FIG. 1 illustrates a system 100 configured in accordance with an embodiment of the invention. The system 100 includes a set of patient machines 102_1 through 102_N in communication with a virtual reality behavioral health platform 104 via a network 106, which may be any combination of wired and wireless networks. Also connected to the network 106 is a set of therapist machines 150_1 through 150_N. The virtual reality behavioral health platform 104 coordinates virtual reality healthcare sessions between therapists operating machines 150_1 through 150_N and patients operating machines 102_1 through 102_N.

Each client machine, e.g. 102_1, includes a central processing unit or processor 110 connected to input/output devices 112 via a bus 114. The input/output devices may include a keyboard, mouse, touch display, a virtual reality headset and the like. A network interface circuit 116 is also connected to the bus 114 to provide connectivity to network 106. A memory 120 is also connected to the bus 114. The memory 120 stores a patient module 122. The patient module 122 includes instructions executed by processor 110 to coordinate interactions with virtual reality behavioral health platform 104, as discussed below.

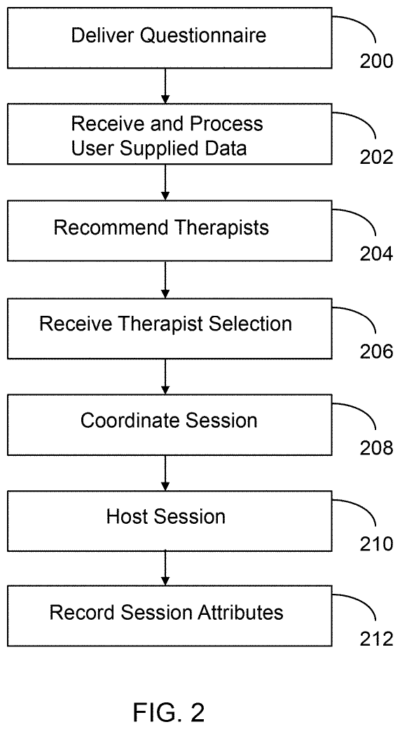

Virtual reality behavioral health platform 104 includes a processor 130, input/output devices 132, bus 134 and network interface circuit 136. A memory 140 is connected to the bus 134. The memory 140 stores a virtual reality health platform module 142 to implement operations discussed in connection with FIG. 2.

Each therapist machine, e.g. 150_1, includes a processor 151, input/output devices 152, bus 154 and network interface circuit 156. A memory 160 is connected to bus 154. The memory 160 stores a therapist module 162 with instructions executed by processor 151 to coordinate interactions with virtual reality behavioral health platform 104, as discussed below.

FIG. 2 illustrates operations implemented by virtual reality behavioral health platform 104. The virtual reality health platform module 142 delivers a questionnaire 200 to one or more client machines 102_1 through 102_N. In one embodiment, the questionnaire is an interactive form that prompts a patient to answer behavioral health questions. In one embodiment, the questionnaire is the Adverse Childhood Experience (ACE) questionnaire developed by Kaiser Permanente and the Center for Disease Control and Prevention.

Preferably, each client machine 102_1 through 102_N and communication sessions with the platform 104 comply with Health Insurance Portability and Accountability Act (HIPAA) standards. An embodiment of the invention utilizes encrypted communication channels for all communications.

The virtual reality health platform module 142 receives and processes user supplied data 202. In particular, the virtual reality health platform module 142 receives user supplied data and automatically computes a health risk score. If the health risk score exceeds a specified threshold, emergency procedures may be automatically invoked, such as coordinating with a therapist to immediately contact a patient. Processing may also include utilizing a matching algorithm to identify qualified therapists for a patient with a given set of behavioral health issues.

Therapists are recommended 202. In particular, a list of therapists is delivered from the virtual reality health platform module 142 to one or more client devices 102_1 through 102_N via network 106. A therapist selection is then received 206 for a patient. The therapist and patient are then linked in a database associated with the virtual reality health platform module 142.

The virtual reality health platform module 142 coordinates a session 208 between a therapist and a patient. In particular, the module 142 provides a scheduling tool that allows a patient or therapist to schedule a session. The module 142 also provides tools that allow the therapist to specify characteristics of the virtual reality environment, such as a beach setting, a café, or a park. The module 142 also provides tools that allow the patient to specify characteristics of the virtual reality environment, such as tools to select an avatar representing the patient and an avatar representing the therapist.

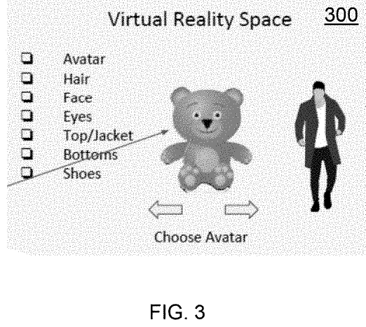

FIG. 3 illustrates an interface 300 for selecting avatars. The arrows indicate that a variety of avatars are available for selection. Moreover, as indicated by the text on the left side of the figure, each avatar can be customized with such parameters as hair, face, eyes, top, bottom and shoes. The utilization of patient selected avatars facilitates cognitive, emotional, behavioral and neurological change in the patient.

The session may transpire at a website, in a mobile application, or in a chat application. The virtual reality environment may be presented on a computer display, mobile device display or virtual reality headset. In one embodiment, the session is assigned a unique room identifier to connect the therapist and the patient, where the unique room identifier is a virtual room hosted by the virtual reality health platform module 142.

Next, a session is hosted 208. In particular, the virtual reality health platform module 142 hosts a session between a patient utilizing a patient machine 102_1 and a therapist utilizing a therapist machine 150_1. The session transpires in a virtual reality scene designed to be secure. In one embodiment, both the patient and the therapist are represented with avatars. The audio on each of the patient machine 102_1 and therapist machine 150_1 is enabled so there can be real time audio communication. In one embodiment, the virtual reality scene is optimized for the patient's disorder, such as one virtual reality scene for depression, a second virtual reality scene for anxiety, etc. The virtual reality scene may be augmented with therapeutic sound, such as waves breaking or birds singing. Preferably, the scene selection is specified by the therapist during step 208 when the session is coordinated.

After the session is hosted, session attributes are recorded 212 by the virtual reality health platform module 142.

In one embodiment, the patient wears a biometric device that allows the therapist to collect heart rate variability readings for each session. Such information can be tracked over time to optimize the treatment plan for the patient.

An embodiment of the invention supports video conferencing between the patient and the therapist. This would typically transpire after initial virtual sessions give the patient confidence for interactions in a video conference.

An embodiment of the present invention relates to a computer storage product with a computer readable storage medium having computer code thereon for performing various computer-implemented operations. The media and computer code may be those specially designed and constructed for the purposes of the present invention, or they may be of the kind well known and available to those having skill in the computer software arts. Examples of computer-readable media include, but are not limited to: magnetic media such as hard disks, floppy disks, and magnetic tape; optical media such as CD-ROMs, DVDs and holographic devices; magneto-optical media; and hardware devices that are specially configured to store and execute program code, such as application-specific integrated circuits (“ASICs”), programmable logic devices (“PLDs”) and ROM and RAM devices. Examples of computer code include machine code, such as produced by a compiler, and files containing higher-level code that are executed by a computer using an interpreter. For example, an embodiment of the invention may be implemented using JAVA®, C++, or other object-oriented programming language and development tools. Another embodiment of the invention may be implemented in hardwired circuitry in place of, or in combination with, machine-executable software instructions.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A machine, comprising:
a processor;
a network interface circuit connected to the processor and a network; and
a memory connected to the processor, the memory storing instructions executed by the processor to:
deliver over the network a questionnaire to a patient machine connected to the network,
receive from the patient machine via the network user supplied data entered by a patient,
process the user supplied data to derive a list of therapists,
deliver via the network the list of therapists to the patient machine,
receive from the patient machine a selected therapist,
coordinate a session between the patient and the selected therapist, wherein the selected therapist utilizes a therapist machine connected to the network to specify virtual reality parameters for the session, and wherein the patient specifies at least one avatar for the session, and
host the session in accordance with the virtual reality parameters, such that the session transpires in a virtual reality environment without identifying the real life visual attributes of the patient or selected therapist, while utilizing audio communications between the patient and the selected therapist.

2. The machine of claim 1 wherein all communications with the patient and selected therapist comply with Health Insurance Portability and Accountability Act (HIPAA) standards.

3. The machine of claim 1 wherein the questionnaire is an Adverse Childhood Experience (ACE) questionnaire.

4. The machine of claim 1 further comprising recording session attributes.

5. The machine of claim 4 wherein the session attributes include biometric data collected from the patient during the session.
